(19) United States
(12) Patent Application Publication
(10) Pub. No.: US 2011/0060358 A1
(43) Pub. Date: Mar. 10, 2011

(54) METHODS AND IMPLANTS FOR INDUCING SATIETY IN THE TREATMENT OF OBESITY

(76) Inventors: Michael J. Stokes, Cincinnati, OH (US); Jason L. Harris, Mason, OH (US); Mark S. Zeiner, Mason, OH (US); Elliott J. Fegelman, Cincinnati, OH (US); William B. Weisenburgh, II, Maineville, OH (US); Christopher J. Hess, Cincinnati, OH (US)

(21) Appl. No.: 12/554,006

(22) Filed: Sep. 4, 2009

(51) Int. Cl. A61M 29/00

(52) U.S. Cl. 606/192; 604/96.01

(57) ABSTRACT

A device for inducing satiety including an elongated device for insertion through a natural orifice and into a stomach of the patient. The distal end of the device includes a means for occupying space between the submucosal and muscularis layers adjacent a pyloric sphincter. The means has a collapsed state for delivery to a target site and an expanded state for implantation thereof.

### FIELD OF THE INVENTION

The present invention relates generally to obesity surgery.

### BACKGROUND OF THE INVENTION

Obesity is a medical condition affecting more than 30% of the population in the United States. Obesity affects an individual's personal quality of life and contributes significantly to morbidity and mortality. Obese patients, i.e. individuals having a body mass index (“BMI”) greater than 30, often have a high risk of associated health problems (e.g., diabetes, hypertension, and respiratory insufficiency), including early death. With this in mind, and as those skilled in the art will certainly appreciate, the monetary and physical costs associated with obesity are substantial. In fact, it is estimated the costs relating to obesity are in excess of 100 billion dollars in the United States alone. Studies have shown that conservative treatment with diet and exercise alone may be ineffective for reducing excess body weight in many patients.

Bariatrics is the branch of medicine that deals with the control and treatment of obesity. A variety of surgical procedures have been developed within the bariatrics field to treat obesity. The most common currently performed procedure is the Roux-en-Y gastric bypass (RYGB). This procedure is highly complex and is commonly utilized to treat people exhibiting morbid obesity. In a RYGB procedure a small stomach pouch is separated from the remainder of the gastric cavity and attached to a resectioned portion of the small intestine. This resectioned portion of the small intestine is connected between the “smaller” gastric cavity and a distal section of small intestine allowing the passage of food therebetween. The conventional RYGB procedure requires a great deal of operative time. Because of the degree of invasiveness, post-operative recovery can be quite lengthy and painful. Still more than 100,000 RYGB procedures are performed annually in the United States alone, costing significant health care dollars.

In view of the highly invasive nature of the RYGB procedure, other less invasive procedures have been developed. These procedures include gastric banding, which constricts the stomach to form an hourglass shape. This procedure restricts the amount of food that passes from one section of the stomach to the next, thereby inducing a feeling of satiety. A band is placed around the stomach near the junction of the stomach and esophagus. The small upper stomach pouch is filled quickly, and slowly empties through the narrow outlet to produce the feeling of satiety. In addition to surgical complications, patients undergoing a gastric banding procedure may suffer from esophageal injury, spleen injury, band slippage, reservoir deflation/leak, and persistent vomiting. Other forms of bariatric surgery that have been developed to treat obesity include Fobi pouch, bilio-pancreatic diversion and gastroplasty or “stomach stapling”.

Morbid obesity is defined as being greater than 100 pounds over one's ideal body weight. For individuals in this category, RYGB, gastric banding or another of the more complex procedures may be the recommended course of treatment due to the significant health problems and mortality risks facing the individual. However, there is a growing segment of the population in the United States and elsewhere who are overweight without being considered morbidly obese. These persons may be 20-30 pounds overweight and want to lose the weight, but have not been able to succeed through diet and exercise alone. For these individuals, the risks associated with the RYGB or other complex procedures often outweigh the potential health benefits and costs. Accordingly, treatment options should involve a less invasive, lower cost solution for weight loss.

With the foregoing in mind, it is desirable to have a surgical weight loss procedure that is inexpensive, with few potential complications, and that provides patients with a weight loss benefit while buying time for the lifestyle changes necessary to maintain the weight loss. Further, it is desirable that the procedure be minimally invasive to the patient, allowing for a quick recovery and less scaring. The present invention provides such a procedure.

### BRIEF DESCRIPTION OF THE DRAWINGS

FIG. 1 is a perspective view of a needle assembly penetrating the mucosal layer of the gastric cavity;

FIG. 2 is a perspective view similar to FIG. 1, showing a fluid being infused between the mucosal and muscularis layers of the gastric wall;

FIG. 3 is a perspective view similar to FIG. 2, showing a fluid pocket formed within the gastric wall;

FIG. 4 is a perspective view of a first embodiment of an implantable device;

FIG. 5 is a schematic view of a needle tip penetrating the gastric wall;

FIG. 6 is a schematic view of a stent device being ejected through the tip of a needle assembly;

FIG. 7 is a schematic view showing the stent device fully implanted under the mucosal layer of the gastric wall;

FIG. 8 is a schematic view showing the mucosal and muscularis layers deformed by the implanted stent;

FIG. 9 is a diagrammatic view of a gastric cavity containing a ring of implanted devices;

FIG. 10 is a perspective view of a spherical stent being deployed from a needle assembly;

FIG. 11 is a perspective view of a gastric cavity, partially cut away to show a stent device implanted in the anterior antrum wall;

FIG. 12 is a perspective view showing a second embodiment for an implantable device being implanted into the gastric wall;

FIG. 13 is a perspective view showing the second implant embodiment being inflated to deform the cavity wall;

FIG. 14 is a perspective view showing a third embodiment for an implantable device being introduced into a gastric wall;

FIG. 15 is a perspective view of the third implant device embodiment, showing the device encircling within the gastric wall;

FIG. 16 is a perspective view similar to FIG. 15, showing an additional length of the wire bunching up within the gastric wall;

FIG. 17 is a perspective view of a gastric cavity, showing a fourth embodiment for an implantable device being implanted into the antrum cavity wall;

FIGS. 18A-18C show expansion of the fourth implantable device during release from a needle assembly;

FIGS. 19A-19C show expansion of the fourth implantable device beneath the mucosal layer of the gastric wall; and

FIG. 20 shows a plurality of the fourth embodiment devices implanted about the periphery of the antrum.

### DETAILED DESCRIPTION OF THE INVENTION

Smooth muscle tumors of the stomach, also known as “stromal cell tumors”, typically originate in the smooth musculature of the gastric wall. Through clinical studies, it has been determined that when stromal cell tumors occur in the antrum and, particularly, in the anterior wall of the antrum, the tumors interrupt the normal contractions of both the circular and longitudinal bands of muscles within the gastric cavity wall. This interruption in muscular contractions slows stomach emptying, resulting in a loss of appetite.

The present invention provides a method for treating obesity which simulates the effects of a stomach cell tumor in order to disrupt and slow gastric emptying. In the present invention, one or more devices are implanted between the mucosal and muscularis layers of the gastric cavity wall to disrupt the normal gastro-muscular movements. The devices may be implanted transesophageally in a minimally invasive procedure using a conventional endoscope with an optical viewing device. Alternatively, the devices may be implanted exogastrically in a minimally invasive laparoscopic procedure. The clinical effect of the implants will be to increase the time the patient feels satiated after eating, thereby decreasing the need and desire to eat, and reducing the overall caloric intake of the patient.

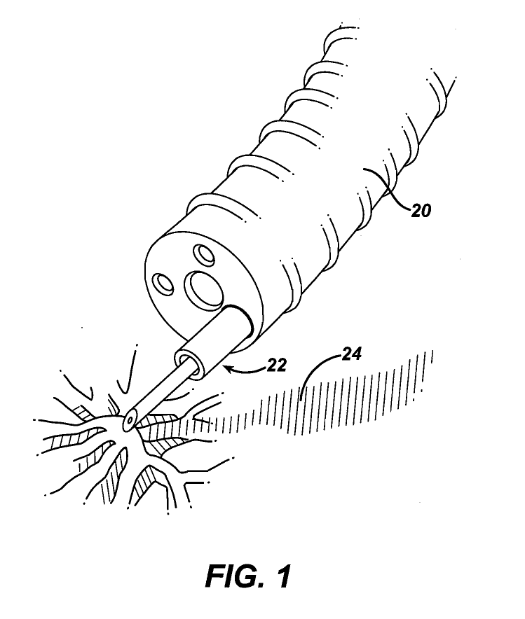

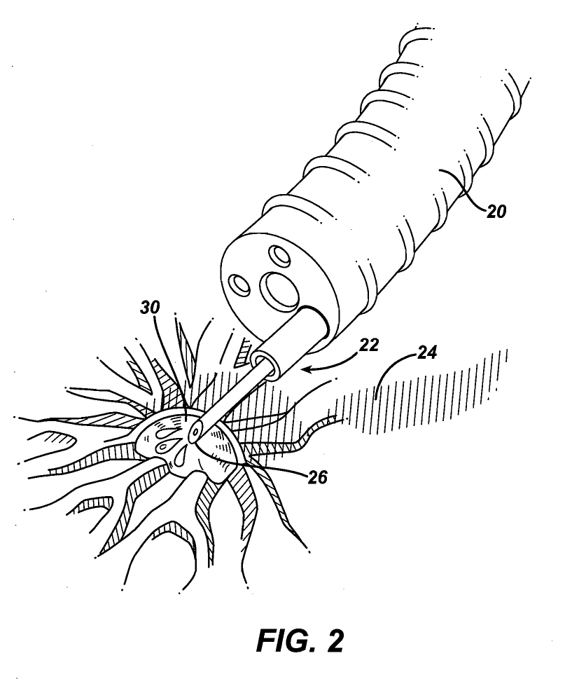

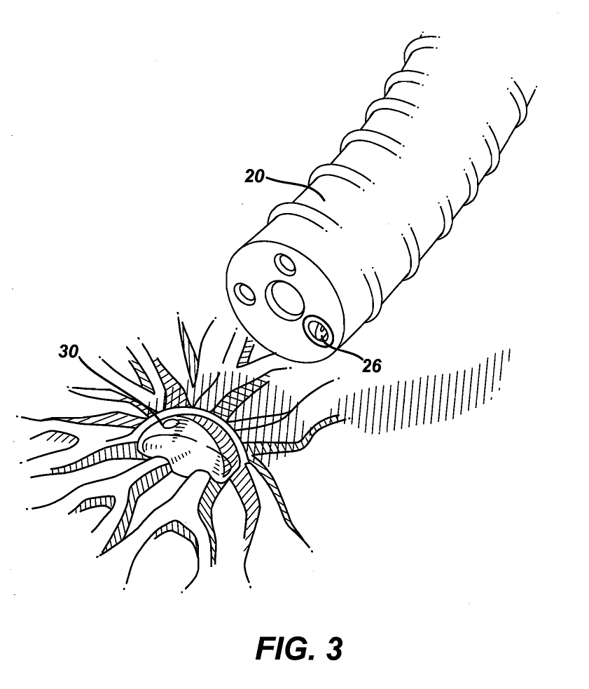

Methods of implanting different device embodiments will now be described using a transesophageal procedure. With an endoscope 20 inserted transorally into the stomach cavity, a needle assembly is passed through the endoscope to the intended location of the implant. To produce optimum results, the implant is placed in the antrum portion of the stomach. Using the needle assembly 22, as shown in FIG. 1, the mucosal layer 24 is penetrated with the needle tip at the intended implant location. With the needle tip 26 between the mucosal and muscularis layers, a fluid is injected through the needle, as shown in FIG. 2, to separate the cavity wall layers and form a fluid pocket or bleb 30 therebetween. Following the infusion of fluid, needle tip 26 is withdrawn from the mucosa 24, as shown in FIG. 3. The needle assembly is then removed from endoscope 20 and replaced with a second needle assembly. This second needle assembly includes an implant device loaded within a needle lumen.

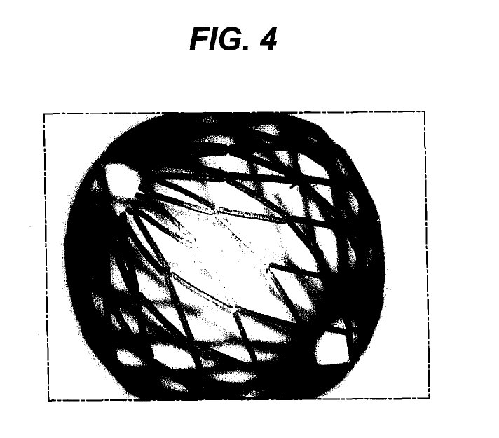

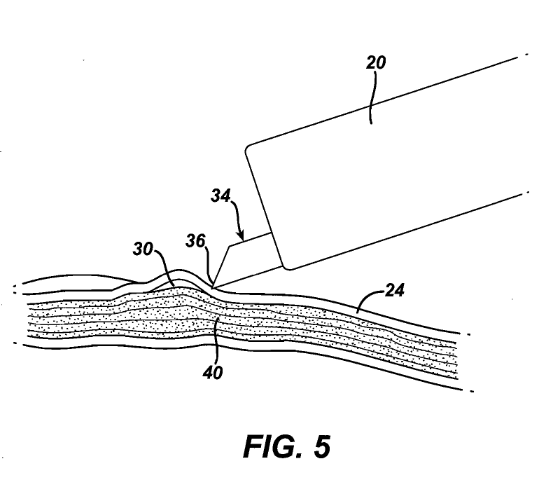

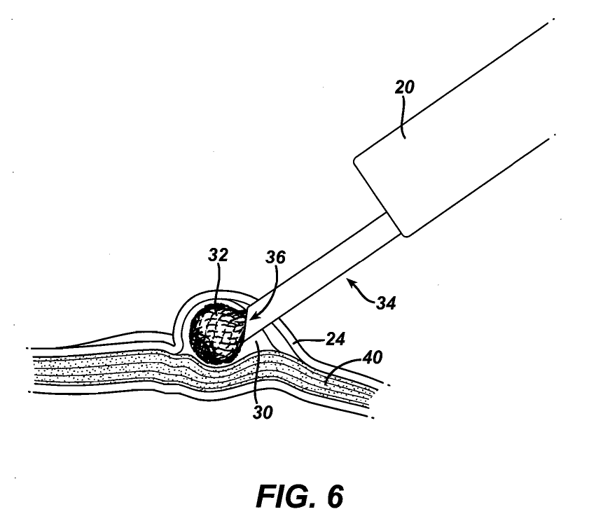

FIG. 4 shows a first embodiment for an implantable device of the present invention. In this embodiment, the device comprises an expandable stent 32 composed of Nitinol, or another type of self-expanding, biocompatible material. In this embodiment, stent 32 is passed through a needle lumen in a compressed form, and then expanded into a spherical shape once implanted within the gastric wall. As shown in FIG. 5, to implant stent 32, second needle assembly 34 is passed through endoscope 20. The sharpened tip 36 of the needle assembly is maneuvered into contact with the mucosal layer 24 of the stomach at the location of bleb 30. Tip 36 of the needle pierces mucosal layer 24, so as to position the distal opening of the needle lumen inside of bleb 30. With needle tip 36 between mucosal layer 24 and muscularis layer 40, stent 32 is passed out of the needle lumen and into the pocket formed between the cavity layers. As stent 32 exits needle 34, the stent expands into a ball-like shape. The expanded stent 32 deforms the surrounding mucosal and muscularis layers, as shown in FIG. 6.

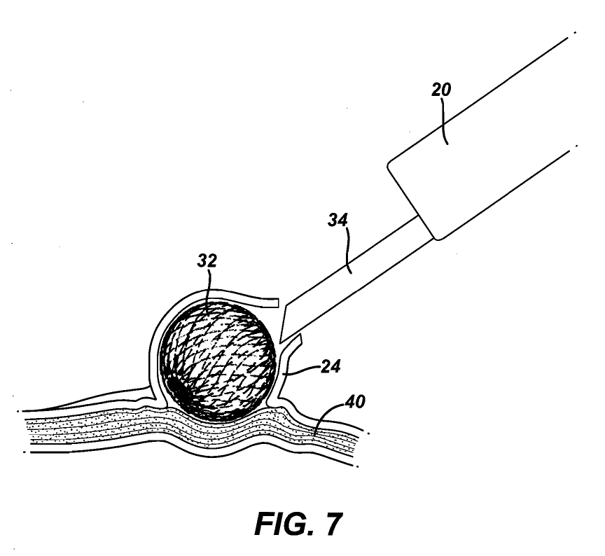

After stent 32 is released, needle tip 36 is removed from the cavity wall, as shown in FIG. 7, and needle assembly 34 retracted back through endoscope 20.

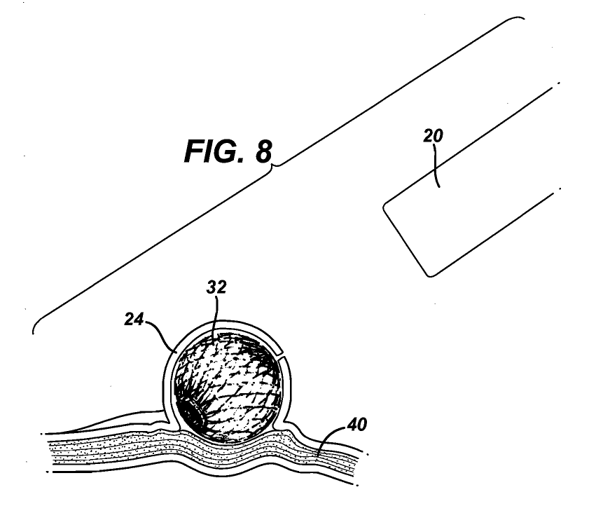

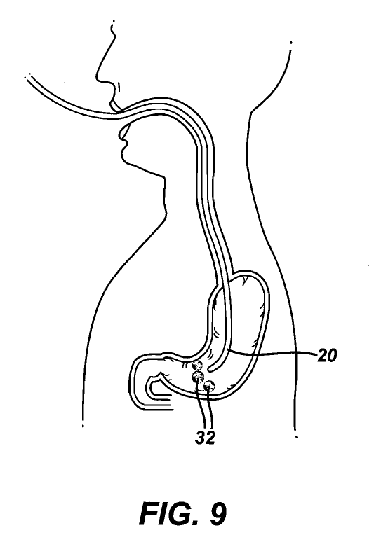

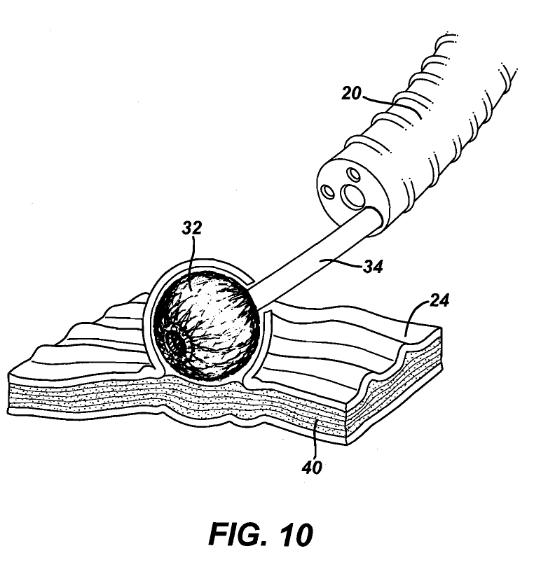

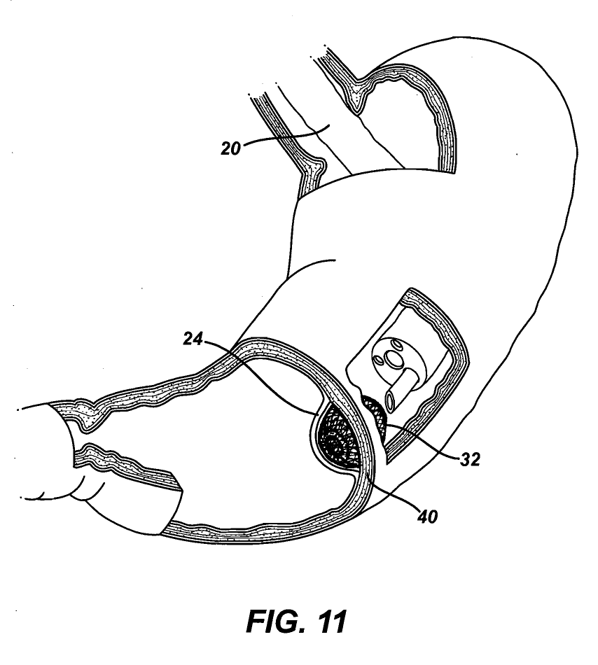

The opening in mucosal layer 24 then closes around stent 32, as shown in FIG. 8. This process of forming a bleb and inserting a stent may be repeated at one or more additional locations in the gastric cavity wall to implant additional stents. The additional stents may also be placed into the anterior wall of the antrum. Alternatively, the additional stents may be placed in a ring about the anterior and posterior walls of the antrum, as shown in FIG. 9. FIGS. 10 and 11 provide additional views of an implanted stent 32, showing the various layers within the gastric cavity wall, and the location of the stent between the mucosal and smooth muscle layers 24, 40. The mesh-type structure of stent 32 promotes tissue ingrowth after implantation, inhibiting migration of the device within or out of the cavity wall.

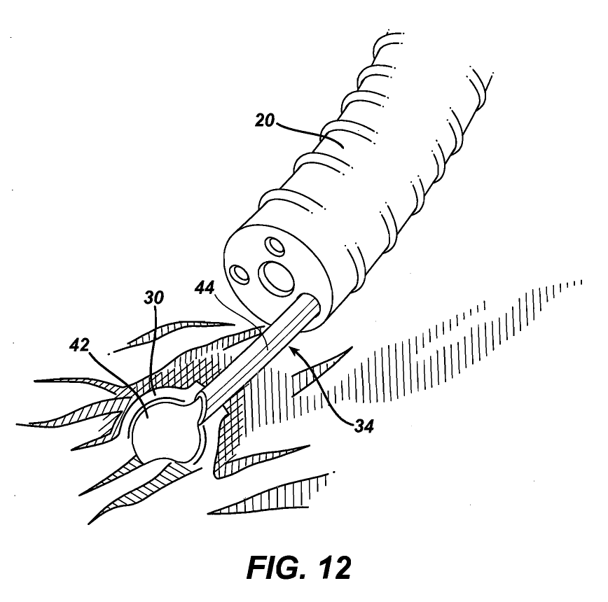

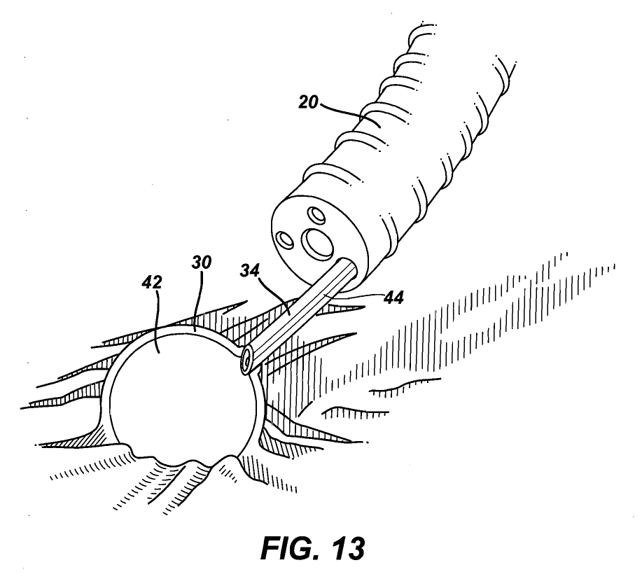

FIG. 12 shows an alternative embodiment for an implantable device, in which the device comprises an inflatable balloon 42. Balloon 42 may be comprised of any bio-compatible material. As shown in FIG. 12, balloon 42 may be inserted via needle assembly 34 into the bleb 30 formed between the mucosal and muscularis layers. A catheter 44 extends through the needle lumen and through an opening in balloon 42. After balloon 42 is inserted into bleb 30, the balloon may be inflated via a fluid passed through catheter 44, as shown in FIG. 13. After balloon 42 is inflated, catheter 44 is removed from the balloon, and the catheter and needle assembly are retracted back through endoscope 20. Additional balloons 42 may be implanted into the anterior antrum wall, or into other locations about the antrum, to achieve the desired effect on the gastric muscular contractions.

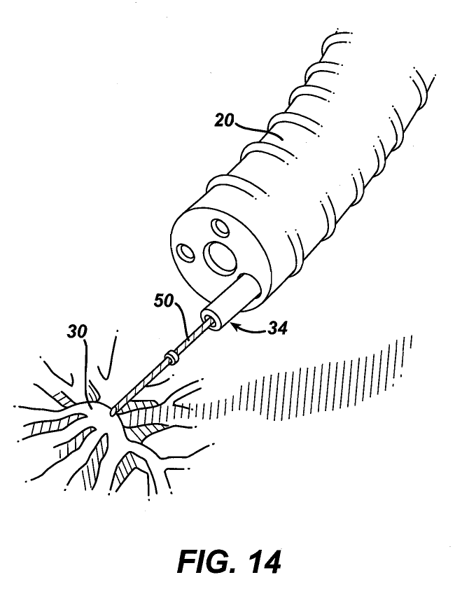

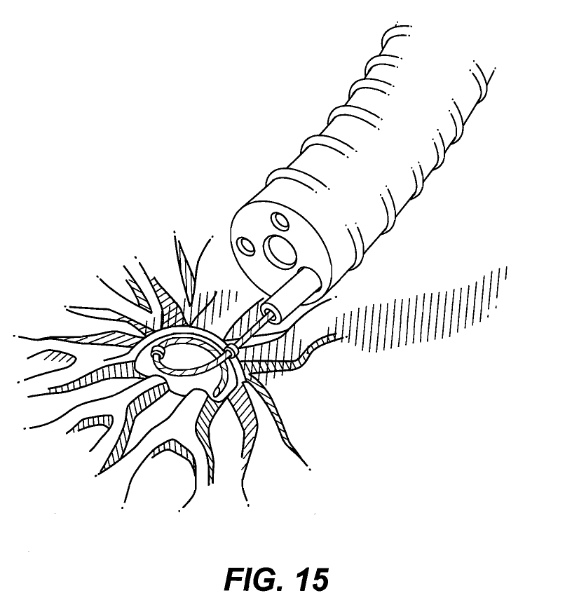

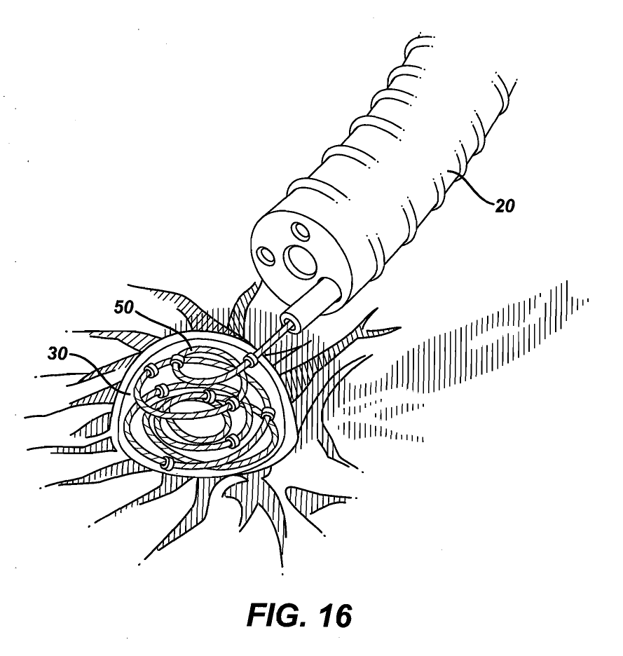

FIG. 14 shows a third embodiment for an implantable device in which the device comprises a length of thin, flexible material such as, for example, a biocompatible metal wire 50. As in the embodiments above, wire 50 may be inserted via needle assembly 34 into the bleb 30 formed between the mucosal and muscularis layers. The tip of needle 34 penetrates the mucosal layer to provide an opening for injecting wire 50 into bleb 30. As the length of wire 50 is passed into the gastric wall, as shown in FIG. 15, the wire is encircled about within bleb 30 to create a bunching effect, and thereby form a three-dimensional implant of increased spatial size. The disoriented arrangement of the encircled wire 50, shown in FIG. 16, inhibits migration of the wire within the gastric layers to maintain the position of the implant. Wire 50 may be formed of Nitinol, titanium, or another type of semi-flexible, biocompatible material. As in the previous embodiments, a plurality of wire lengths 50 may be implanted at various locations within the antrum to achieve the desired effect on the gastric cavity.

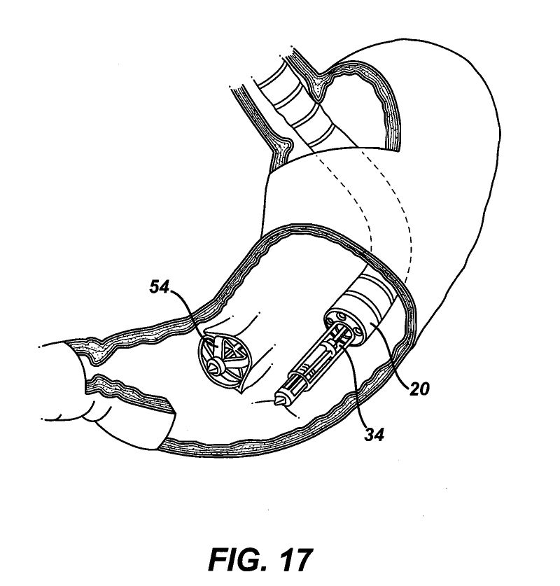

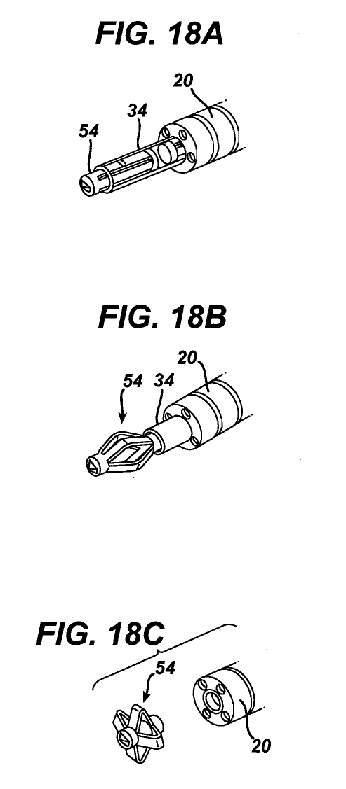

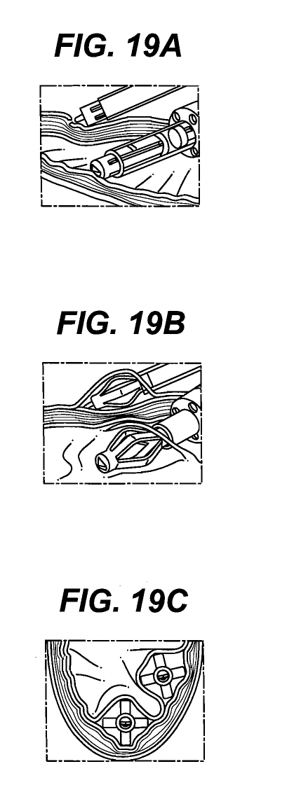

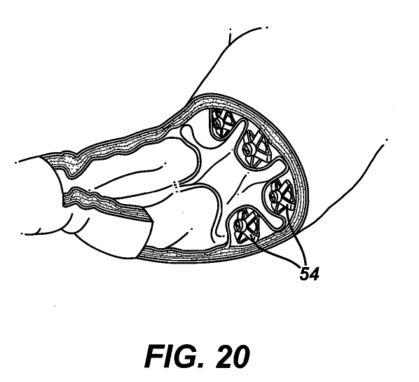

FIG. 17 shows a fourth embodiment for an implantable device in which the device comprises a molly bolt 54. Bolt 54 has a compressed shape, shown in FIGS. 18A and 19A, during entry through needle assembly 34 and mucosal layer 24. As bolt 54 is released into bleb 30, the sides of the bolt expand outward, as shown in FIGS. 18B and 19B. Once bolt 54 is fully released from needle assembly 34, the bolt assumes a maximum spatial capacity, as shown in FIGS. 18C and 19C. The expanded size of bolt 54 within bleb 30 allows the bolt to deform the surrounding areas of the cavity wall. A ring of bolts 54 may be formed around the antrum, as shown in FIG. 20, to produce deformation of the gastric layers about the perimeter of the cavity.

As described above, the implant devices of the present invention can vary as to shape and composition, with the goal that the implant interferes with the contraction of the longitudinal and circular gastric muscles during digestion. The devices' interference with the normal muscle contractions increases gastric emptying times and, thus, prolongs the feeling of satiety. Each of the implants described above is formed of a bio-compatible material that resists migration within the stomach wall. Any number of the devices may be implanted during a procedure, depending upon the desired degree of muscular disruption.

The foregoing description of preferred embodiments of the invention has been presented for purposes of illustration and description. It is not intended to be exhaustive or to limit the invention to the precise form disclosed. Obvious modifications or variations are possible in light of the above teachings. The embodiments were chosen and described in order to best illustrate the principles of the invention and its practical application to thereby enable one of ordinary skill in the art to best utilize the invention in various embodiments and with various modifications as are suited to the particular use contemplated. It is intended that the scope of the invention be defined by the claims appended hereto.

What is claimed:

1. A device for inducing satiety, said device comprising:
a. an elongated device for insertion through a natural orifice and into a stomach of the patient;
b. a distal end of said device includes a means for occupying space between the submucosal and muscularis layers adjacent a pyloric sphincter, said means having a collapsed state for delivery to a target site and an expanded state for implantation thereof.

2. The device of claim 1 wherein said means for occupying space between the submucosal and muscularis layers adjacent a pyloric sphincter expands in volume from its collapsed state to its expanded state.

3. The device of claim 2 wherein said means for occupying space between the submucosal and muscularis layers adjacent a pyloric sphincter comprises a self-expanding biocompatible material.

4. The device of claim 1 wherein said means for occupying space between the submucosal and muscularis layers adjacent a pyloric sphincter comprises a mesh.

5. A device for inducing satiety, said device comprising:
a. an elongated device for insertion through a natural orifice and into a stomach of the patient;
b. said distal end of said device including a detachable expandable balloon for occupying space between the submucosal and muscularis layers adjacent a pyloric sphincter, said means having a collapsed state for delivery to a target site and an expanded state for implantation thereof.
